(19) United States
(12) Patent Application Publication
(10) Pub. No.: US 2010/0286792 A1
(43) Pub. Date: Nov. 11, 2010

(54) FEMORAL STEM AND ITS TOTAL HIP PROSTHESIS

(75) Inventors: Yan Wang, Beijing (CN); Cheng-Kung Cheng, Beijing (CN)

Correspondence Address: WORKMAN NYDEGGER;1000 Eagle Gate Tower, 60 East South Temple, Salt Lake City, UT, 84111, US

(73) Assignee: NATON INVESTMENT CO., LTD, Beijing (CN)

(21) Appl. No.: 12/811,351

(22) PCT Filed: Feb. 4, 2009
(86) PCT No.: PCT/CN2009/070354

§ 371 Date: Jun. 30, 2010

(30) Foreign Application Priority Data
Dec. 4, 2008 (CN) 200810239001.6

(51) Int. Cl. A61F 2/36; A61F 2/32

(52) U.S. Cl. 623/23.15; 623/22.11

(57) ABSTRACT

This invention provides a femoral stem comprising a neck portion and an anchoring portion, wherein the inner curve of said anchoring portion conforms to the anatomic curve of the corresponding part of AVN patients. This invention also provides a total hip prosthesis embodying said femoral stem. Good morphological fit of said femoral stem geometry against the femoral medullary cavity of AVN patients enhances the long-term stability of prosthesis.

### FIELD OF THE INVENTION

This invention relates to artificial transplantation technology, more particularly, relates to a special femoral stem and its total hip prosthesis for Avascular Necrosis patients.

### BACKGROUND OF THE INVENTION

Avascular Necrosis (AVN) is a common but refractory disease in clinic, caused by the damage of the blood supply to the femoral head which can lead to ischemia, necrosis or, collapse of bone and followed by serious hip dysfunction.

For patients with severe avascular necrosis, total hip replacement is the major treatment method.

To solve the problem that the long-term loosening rate of cemented prostheses in Total Hip Arthroplasty (THA) increased continuously year by year, cementless fixation technique has been introduced in femoral stem installation from 1970s. From 1980s, the technique was applied in the fixation of acetabular component. To provide the initial and long-term stability, various designs of femoral stem have been adopted, but only a small portion of these designs succeed. The intensive research results have showed that the higher morphological fit of the prosthesis geometry to femoral medullary cavity can provide more durable bone ingrowth, and the amount of permitted micromotion at the interface should be controlled less than 28 μm˜50 μm. Therefore, implant geometry design decides the morphological fit degree, even the survival rate of the prosthesis. However, prostheses for THA in market with the fluted surfaces, or modular components or anatomic femoral stems are all designed according to the measured anatomic data from normal westerner population and reported by foreign researchers. For these prostheses, it is difficult to achieve a preferable morphological fit in Chinese population. And the assistant fixation designs for improving the performances of these prostheses, such as surface coatings, fixation screws and pegs, can only act as the secondary fixation methods and provide the limited initial stability. Thus, to achieve long-term stability, enhancing the morphological fit of the prosthesis to the femoral cavity is the necessary way.

Currently, titanium alloy material is still considered to be the best internal fixation materials due to its good biocompatibility and its elastic modulus is more close to that of cortical bone , which can reduce the foreign body reactions and the stress shielding theoretically. But that doesn't indicate the morphological fit of the prosthesis to the femoral cavity is no longer important, whereas it puts forward higher requirements for the fixation of proximal femur, because the elastic modulus of cancellous bone, largely existing in the proximal femoral medullary cavity, is much lower than that of titanium alloy. However, the above mentioned problem is not obtained enough attentions in the present prostheses designs. Clinically, experienced doctors obtain acceptable fit by cutting redundant cancellous bone to fit the implant geometry, which is similar to cutting the feet to fit the shoes.

The morphological fit degree is influenced by the factors including age, race, gender and so on. There is no verdict for whether disease influences the shape of medullary cavity, but the correlation between femoral neck fractures and the shape of femur in AVN patients has been supported by more and more literatures.

Joint replacement caused by Avascular Necrosis almost accounts for 40% in the all joint replacement in China. The ages of AVN patients operated the hip joint replacement are just older than those of Congenital coxarthropathy patients, and much younger than those of osteoarthritis and femoral neck fracture patients. Thus, to realize long-term stability of prosthesis, the same as survival rate of prosthesis, is particularly important.

Clinically, the differences in femoral medullary cavity, femoral anteversion angle and acetabulum characteristics have been investigated via the hip joint radiographies of 186 AVN patients and 213 normal persons in China. And some statistical differences in the anterior, inner and lateral curves of femoral medullary cavity between the two groups are found. Most AVN patients have the “champagne-flute” femoral medullary cavity, consisting with surgical findings. Therefore, it is of great significance to design prostheses according to the morphological characteristics of AVN patients' femoral cavities.

### SUMMARY OF THE INVENTION

The present invention provides a femoral stem prosthesis designed in accordance with the statistically averaged data of femoral cavities of AVN patients to ensure the good morphological fit of the prosthesis to femoral cavity of AVN patients to achieve the long-term stability.

Another object of the present invention is to provide a total hip prosthesis embodying said femoral stem.

Technical Solution of the invention is as follows: A femoral stem comprises a neck portion and an anchoring portion, wherein the inner curve of said anchoring portion is the anatomic curve of the corresponding part of AVN patients.

The equation of the inner curve of said anchoring portion is y =±A₁x³+A₂x²±A₃x+A₄; A₁, A₂, A₃, A₄ are coefficients, and the value ranges of A₁, A₂, A₃ and A₄ are −0.17000˜0.04054, −2.00586˜7.10783, −87.75095˜40.25685 and −246.58554˜338.85144, respectively.

The length of said inner curve is 10%˜100% that of the whole anchoring portion.

The anterior curve of said anchoring portion is the anatomic curve of the corresponding part of AVN patients.

The equation of the anterior curve of said anchoring portion is y=±B₁x³+B₂x²±B₃x+B₄; B₁, B₂, B₃, B₄ are coefficients, and the value ranges of B₁, B₂, B₃ and B₄ are −0.17000˜2.61338, −75.67478˜8.53086, −73.68055˜720.37993 and −3125.92948˜283.55694, respectively.

The length of said anterior curve is 10%˜100% that of the whole anchoring portion.

Said anchoring portion is short handle.

The distal end of said anchoring portion is highly polished.

Said anchoring portion is widely coated except the highly polished distal end.

A total hip prosthesis embodying the femoral stem comprises said femoral stem and an acetabular prosthesis matched with said femoral stem.

Technical effect of the invention is as follows:

The curve of anchoring portion of femoral stem in the present invention is designed in accordance with the inner curve of femoral medullary cavity of AVN patients, based on the statistic average data of femoral geometric characteristics measured in clinical investigation. The femoral stem prosthesis manufactured according to the above curve can ensure higher morphological fit between the femoral medullary cavity of AVN patients and the inner, lateral curves of femoral stem, which may improve the long-term stability. Therefore, this solves the chronically existing problems of lower morphological fit of the prosthesis to the femoral medullary cavity, and lower long-term stability.

The surface of distal end of femoral stem in the present invention is highly polished, which not only can make revisions more conveniently, but also can reduce the stress shielding in distal end. The anchoring portion is widely coated except the highly polished distal end, which can provide the biological fixation, namely osseointegration, by inducing the long-term bone ingrowth.

### BRIEF DESCRIPTION OF THE FIGURES

FIG. 1 is a schematic view of structure of the total hip prosthesis of the present invention;

FIG. 2 is a schematic view of outline of the femoral stem of the present invention;

FIG. 3 is a side view of the femoral stem showed in the FIG. 2.

### DETAILED DESCRIPTION OF THE PREFERRED EMBODIMENT

The present invention will be described more fully regarding on total hip prosthesis hereinafter with reference to the accompanying drawings.

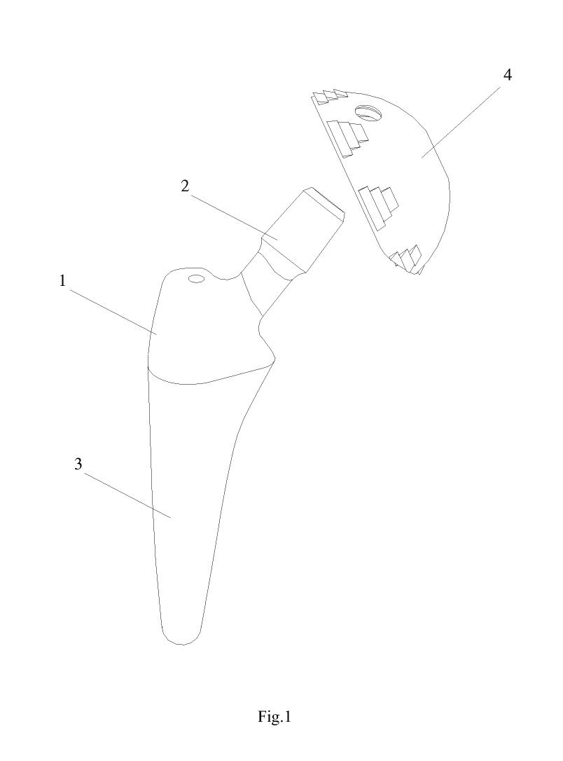

The total hip prosthesis of the present invention shown in FIG. 1 comprises an acetabular cup 4 and a femoral stem 1, and said femoral stem 1 comprises a neck portion 2 and an anchoring portion 3. The acetabular cup 4 articulates with the neck portion 2, and the curve of the anchoring portion 3 of the femoral stem 1 coincides with the anatomic curve of corresponding part of AVN patients.

Said anchoring portion 3 of the femoral stem 1 in the present example is a short handle in favor of reserving more bones in surgery. And its curve coincides with the anatomic curve of the corresponding part of AVN patients.

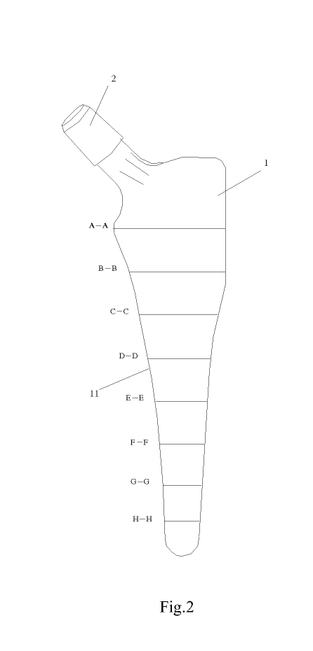

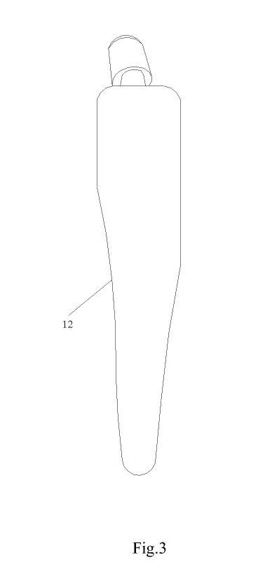

The schematic view of outline and the side view of the femoral stem are respectively shown in FIG. 2 and FIG. 3.

The curve 11 and 12 are the inner and anterior curve of the femoral stem respectively, and their parameters are designed according to the statistic method and are obtained by measuring, counting and processing the femoral skeleton geometric characteristics of AVN patients. In the present example, the femoral curve of AVN patients, differing from that of the normal people, is plotted on several key points using curve fitting, which are determined by the arithmetical equally spaced sections A-A, B-B, C-C, D-D, E-E, F-F, G-G and H-H, etc.

Based on the clinic average data of the femoral geometric characteristics of AVN patients , this present invention defines the equation of the inner curve 11 as: y=±A₁x³+A₂x²±A₃x+A₄; A₁, A₂, A₃, A₄ are coefficients, and the value ranges of A₁, A₂, A₃ and A₄ are −0.17000˜0.04054, −2.00586˜7.10783, −87.75095˜40.25685 and −246.58554˜338.85144, respectively.

The length of inner curve 11 is 10%˜100% of the whole length of the anchoring portion 3.

In FIG. 3, equation of the anterior curve 12 is y=±B₁x³+B₂x²±B₃x+B₄; B₁, B₂, B₃, and B₄ are coefficients, and the value ranges of B1, B2, B3 and B4 are −0.17000˜2.61338, −75.67478˜8.53086, −73.68055˜720.37993 and −3125.92948˜283.55694, respectively.

The length of anterior curve 12 is 10%˜100% of the whole length of the anchoring portion 3.

Furthermore, by the similar design method, the femoral outer and posterior curves located in the vertical direction of said curves 11 and 12, respectively, as well as the femoral anteversion angle and the femoral collodiaphyseal angle, can also be defined, which hardly differ from those of normal persons. Thus, based on the above date, a mathematical model, more similar to the femoral geometric characteristics of AVN patients, can be established to facilitate the manufacturing of total hip prostheses. Compared with the prostheses for THA in the present market, which are manufactured completely according to those statistics data of western people, these total hip prostheses are more adaptive for femoral geometry of AVN patients, and have higher morphological fit degree to the femoral medullary cavity of patients, improving its long-term stability. Therefore, it solves the problems of lower morphological fit of the prosthesis to the femoral medullary cavity, and lower long-term stability.

1. A femoral stem, comprising a neck portion and an anchoring portion, wherein the inner curve of said anchoring portion is the anatomic curve of the corresponding part of AVN patients.

2. A femoral stem of claim 1, wherein the inner curve of said anchoring portion is defined by the equation y=±A₁x³+A₂x²±A₃x+A₄; A₁, A₂, A₃ and A₄ are coefficients, and the value ranges of A1, A2, A3 and A4 are −0.17000˜0.04054, −2.00586˜7.10783, −87.75095˜40.25685 and −246.58554˜338.85144, respectively.

3. A femoral stem of claim 2, wherein the length of said inner curve is 10% to 100% that of the whole anchoring portion.

4. A femoral stem of claim 2, wherein the anterior curve of said anchoring portion is the anatomic curve of the corresponding part of AVN patients.

5. A femoral stem of claim 3, wherein the equation of the anterior curve of said anchoring portion is y=±B₁x³+B₂x²±B₃x+B₄; B₁, B₂, B₃ and B₄ are coefficients, and the value ranges of B1, B2, B3 and B4 are −0.17000˜2.61338, −75.67478˜8.53086, −73.68055˜720.37993 and −3125.92948˜283.55694, respectively.

6. A femoral stem of claim 4, wherein the length of said anterior curve is 10%˜100% that of the whole anchoring portion.

7. A femoral stem of claim 1, wherein said anchoring portion is a short stem.

8. A femoral stem of claim 1, wherein the distal end of said anchoring portion is highly polished.

9. A femoral stem of claim 1, wherein said anchoring portion has a widely coated surface except the highly polished distal end.

10. (canceled)

11. A total hip prosthesis, comprising:
a femoral stem, including a neck portion and an anchoring portion, wherein the inner curve of said anchoring portion is the anatomic curve of the corresponding part of AVN patients; and
a coupled acetabular prosthesis matched with said femoral stem.

12. A total hip prosthesis of claim 11, wherein the inner curve of said anchoring portion is defined by the equation y=±A₁x³+A₂x²±A₃x+A₄; A₁, A₂, A₃ and A₄ are coefficients, and the value ranges of A1, A2, A3 and A4 are −0.17000˜0.04054, −2.00586˜7.10783, −87.75095˜40.25685 and −246.58554˜338.85144, respectively.

13. A total hip prosthesis of claim 12, wherein the length of said inner curve is 10% to 100% that of the whole anchoring portion.

14. A total hip prosthesis of claim 12, wherein the anterior curve of said anchoring portion is the anatomic curve of the corresponding part of AVN patients.

15. A total hip prosthesis of claim 13, wherein the equation of the anterior curve of said anchoring portion is y=±B₁x³+B₂x²±B₃x+B₄; B₁, B₂, B₃ and B₄ are coefficients, and the value ranges of B1, B2, B3 and B4 are −0.17000˜2.61338, −75.67478˜8.53086, −73.68055˜720.37993 and −3125.92948˜283.55694, respectively.

16. A total hip prosthesis of claim 14, wherein the length of said anterior curve is 10%˜100% that of the whole anchoring portion.

17. A total hip prosthesis of claim 11, wherein said anchoring portion is a short stem.

18. A total hip prosthesis of claim 11, wherein the distal end of said anchoring portion is highly polished.

19. A total hip prosthesis of claim 11, wherein said anchoring portion has a widely coated surface except the highly polished distal end.
